(19) United States
(12) Patent Application Publication
(10) Pub. No.: US 2014/0309723 A1
(43) Pub. Date: Oct. 16, 2014

(54) STENT WITH SHEATH AND METAL WIRE

(71) Applicant: INSPIREMD, LTD., Tel Aviv (IL)

(72) Inventors: Eli BAR, Moshav (IL); Asher HOLZER, Raanana (IL); Ofir PAZ, Rishon Lezion (IL)

(21) Appl. No.: 14/315,001

(22) Filed: Jun. 25, 2014

Related U.S. Application Data
(63) Continuation of application No. 13/994,739, filed on Jun. 16, 2013.
(60) Provisional application No. 61/424,650, filed on Dec. 19, 2010.

(51) Int. Cl. A61F 2/07; D04B 1/22

(52) U.S. Cl. CPC A61F 2/07; D04B 1/225; A61F 2002/075
USPC 623/1.13; 29/428; 66/170; 66/190

(57) ABSTRACT

A stent assembly includes a stent and a fiber mesh sleeve covering the stent. A wire runs along the stent over the sleeve and fastened to the stent at multiple points so as to attach the sleeve to the stent.

### CROSS-REFERENCE TO RELATED APPLICATION

This application is a continuation of U.S. patent application Ser. No. 13/994,739, filed Jun. 13, 2013, now pending, which is a national stage of PCT/IB2001/55758, filed Dec. 18, 2011, now expired, which claims the benefit of U.S. Provisional Patent Application 61/424,650, filed Dec. 19, 2010, the entire contents of each of which is incorporated herein by reference.

### FIELD OF THE INVENTION

The present invention relates generally to implantable medical devices, and particularly to vascular implants.

### BACKGROUND OF THE INVENTION

Stents are commonly used in percutaneous coronary revascularization procedures, as well as in treating stenoses in peripheral vasculature, such as in the carotid vessels, organs and extremities. The stent is inserted through the vascular system in a contracted state, until it reaches the desired treatment location, and is then expanded in situ to press outward against the vessel wall. Stents typically comprise a mesh of large apertures, which can damage the surrounding stenotic vessel during expansion and can release dangerous emboli into the bloodstream. The damaged tissue is prone to form new scar tissue, which protrudes through the mesh of the stent and can lead to restenosis.

Some stents are fitted with a tubular, flexible jacket for preventing restenosis and reducing the risk of emboli. For example, PCT International Publication WO 2008/062414, whose disclosure is incorporated herein by reference, describes a stent assembly with a stent jacket, comprising an expandable fiber mesh structure fastened around an expandable stent. When the stent is expanded in a blood vessel, the jacket encourages formation of a stable layer of endothelial cells covering the fibers, while reducing platelet aggregation. WO 2008/062414 shows and describes a number of ways in which the stent jacket can be mounted to the stent, including both adhesive and sliding connections, made by knots.

### SUMMARY

Embodiments of the present invention that are described herein provide improved jackets for implantable devices, and particular improved methods for attachment of a jacket to a device.

There is therefore provided, in accordance with an embodiment of the present invention, a stent assembly, including a stent and a fiber mesh sleeve covering the stent. A wire runs along the stent over the sleeve and fastened to the stent at multiple points so as to attach the sleeve to the stent.

The wire may be point-welded to the stent at the multiple points. In a disclosed embodiment, the stent includes multiple struts, and the wire is fastened to the stent along an outermost strut of the stent. Typically, the wire includes first and second wires, which are fastened to the stent over the sleeve at first and second ends of the stent, respectively, without additional fastening of the sleeve to the stent between the first and second ends.

In one embodiment, the sleeve includes a knit having multiple eyes, and the points at which the wire is fastened to the sleeve are inside the eyes of the knit.

There is also provided, in accordance with an embodiment of the present invention, a method for producing a stent assembly, which includes positioning a fiber mesh sleeve over a stent. A wire is run along the stent over the sleeve and is fastened to the stent at multiple points so as to attach the sleeve to the stent.

The present invention will be more fully understood from the following detailed description of the embodiments thereof, taken together with the drawings in which:

### BRIEF DESCRIPTION OF THE DRAWING

FIG. 1 is a schematic, pictorial illustration of a stent assembly, in accordance with an embodiment of the present invention.

### DETAILED DESCRIPTION OF EMBODIMENTS

The inventors have found that for optimal performance of a stent jacket of the type described above, it is desirable that the jacket be mounted on the stent in a manner that allows the fibers of the jacket to slide over the stent as the stent expands. Embodiments of the present invention provide means and methods for such mounting.

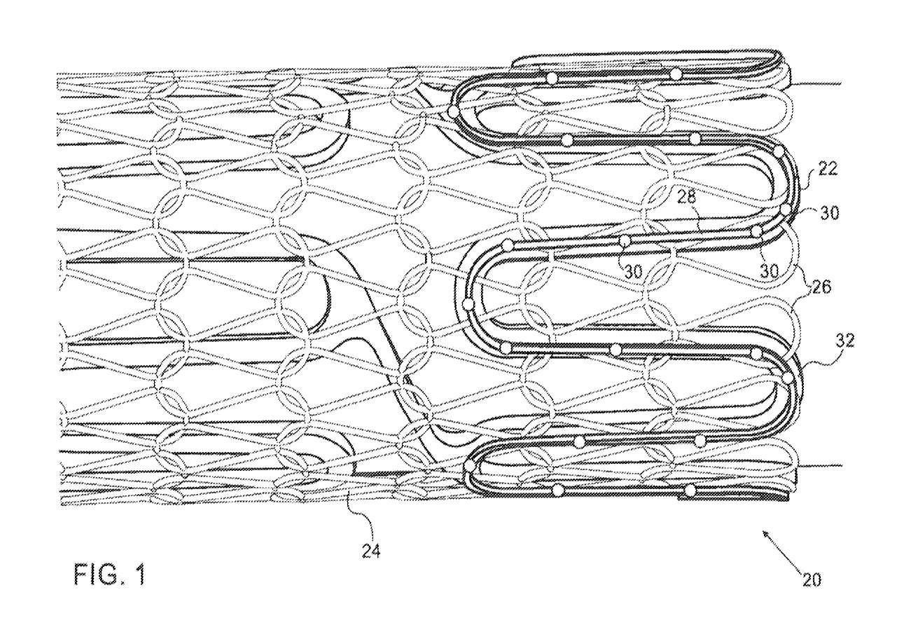

FIG. 1 is a schematic, pictorial illustration showing one end of a stent assembly 20 of this sort, in accordance with an embodiment of the present invention. Assembly 20 comprises a stent 22 made from a flexible, biocompatible material, typically a metal alloy such as stainless steel or Nitinol, as is known in the art. A fiber mesh sleeve 24 covers stent 22. The mesh in this embodiment is a knit comprising interlocking eyes 26, which define pores of a desired size when the stent assembly is expanded inside a blood vessel. It is desirable that all the eyes at the ends of sleeve 24 be fastened to stent 22 in order to prevent the knit from unraveling or sliding off the stent.

After aligning sleeve 24 with stent 22, a metal wire 28 is used to attach the sleeve to the stent. Wire 28 is typically made from a biocompatible metal, such as stainless steel, Nitinol, CoCr or any other suitable biocompatible metal alloy, and has a diameter in the range of 10-100 microns. As shown in the figure, wire 28 runs along an outermost strut 32, at the end of stent 22, over the fibers of sleeve 24. The wire is fastened to the radial surface of the underlying strut, typically by multiple point-welds 30, which may be produced by a laser micro-welder, for example. Alternatively, other methods of metal-to-metal fastening may be used. Typically, to hold the sleeve securely and prevent unraveling, one point-weld is made inside each eye 26 of the sleeve that overlies the strut. A similar welded-wire connection is made at the opposite end of the stent assembly (not shown). To allow the sleeve to slide freely over the stent during expansion of the stent, there is typically no additional fastening of the sleeve to the stent between the two ends.

Alternatively, other weld patterns may be used. For example, point-welds 30 may be more closely spaced, with two or more point-welds in at least some of eyes 26. On the other hand, the point-welds may be more widely spaced, at least in some locations, skipping over at least some of the eyes.

The point-welding technique illustrated in FIG. 1 is advantageous in that it fastens sleeve 24 to stent 22 quickly, conveniently, and securely. Between point-welds 30, however, the fibers of sleeve 24 are free to move between wire 28 and stent 22. Therefore, as stent assembly 20 expands, the fibers of sleeve 24 are able to shift relative to the stent in order to maintain relatively uniform pore sizes.

In an alternative embodiment (not shown in the figures), wire 28 runs along the axial, external edge of the outer strut of stent 22 and is point-welded to the edge, rather than to the radial surface as shown in FIG. 1. Further alternatively, other wire and weld configurations may be used and are considered to be within the scope of the present invention. For example, a wire may be placed over the sleeve and welded to the stent at the center of the stent or at some other location between the ends of the stent, in addition to or instead of the wires used at the ends of the stent as shown in the figure.

It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

What is claimed is:

1. A stent assembly, comprising:
a stent comprising a biocompatible metal alloy;
a fiber mesh sleeve covering the stent and defining a plurality of pores between the fibers; and
a wire, running along the stent and fastened to the stent and the sleeve at multiple points so as to slidably attach the sleeve to the stent.

2. The stent assembly of claim 1, wherein the fiber mesh sleeve is a knit structure to which the wire is slidably fastened at a plurality of the multiple points.

3. The stent assembly of claim 1, wherein the stent comprises multiple struts, and wherein the wire is fastened to the stent along an outermost strut of the stent.

4. The stent assembly of claim 3, wherein the wire comprises first and second wires, which are fastened to the stent over the sleeve at first and second ends of the stent, respectively, without additional fastening of the sleeve to the stent between the first and second ends.

5. The stent assembly of claim 1, wherein the sleeve comprises a knit structure having multiple eyes, and wherein the points at which the wire is fastened to the sleeve are inside the eyes of the knit.

6. The stent assembly of claim 5, wherein the knit structure comprises interlocking eyes.

7. The stent assembly of claim 5, wherein the eyes are fastened to the stent to prevent the knit sleeve from unraveling or sliding off the stent.

8. The stent assembly of claim 1, wherein the wire is fastened by at least two points in a plurality of the eyes.

9. The stent assembly of claim 1, wherein the fibers of the sleeve have relatively uniform size pores over the sleeve when the stent is in a retracted position and when the stent is in an expanded position.

10. The stent assembly of claim 1, wherein the wire is point-welded to an edge of the stent.

11. The stent assembly of claim 1, wherein the wire comprises a diameter of 10 to 100 microns.

12. A method for producing a stent assembly, which comprises:
positioning a fiber mesh sleeve over a stent;
running a wire along the stent over the sleeve; and
fastening the wire to the stent at multiple points so as to slidably attach the sleeve to the stent as the stent expands from a retracted position to a radially expanded position.

13. The method of claim 12, wherein the stent comprises multiple struts, and wherein the wire is fastened to the stent along an outermost strut of the stent.

14. The method of claim 13, wherein fastening the wire comprises attaching first and second wires to the stent over the sleeve only at first and second ends of the stent, respectively, without additional fastening of the sleeve to the stent between the first and second ends.

15. The method of claim 12, which further comprises knitting the fibers to form the fiber mesh sleeve.

16. The method of claim 15, wherein the knit sleeve is selected to comprise multiple eyes, and wherein a plurality of the points at which the wire is attached are inside the eyes of the knit.

17. The method of claim 15, wherein the knitting comprises providing interlocking eyes to the fiber.

18. The method of claim 16, wherein the eyes are fastened to the stent with the wire to prevent the knit sleeve from unraveling or sliding off the stent.

19. The method of claim 12, wherein the wire is fastened by at least two points in a plurality of the eyes.

20. The method of claim 12, wherein the fibers of the sleeve are sized to have relatively uniform pores over the sleeve while the attached stent is radially expanded.

21. The method of claim 12, wherein the wire is attached to a plurality of eyes at each terminal end of the sleeve.

22. The method of claim 12, wherein the wire is selected to comprise a diameter of 10 to 100 microns.

23. A stent assembly, comprising:
a stent comprising a flexible, biocompatible metal alloy and multiple struts, where;
a knit fiber mesh sleeve covering the stent and defining a plurality of pores between the fibers; and
a wire having a diameter of 10 to 100 microns, running along the stent over the sleeve and fastened along an outermost strut of the stent, and being fastened to the stent at multiple points each within a plurality of the pores so as to slidably attach the sleeve to the stent so as to maintain relatively uniform pore sizes across the sleeve when the stent is in a retracted position and in an expanded position.
